(19) United States
(12) Patent Application Publication
(10) Pub. No.: US 2010/0217133 A1
(43) Pub. Date: Aug. 26, 2010

(54) ELECTROCARDIOGRAM DERIVED APNOEA/HYPOPNEA INDEX

(75) Inventors: Kris Nilsen, Fitzroy North (AU); Eugene Zilberg, Sandringham (AU)

Correspondence Address: SUGHRUE MION, PLLC, 2100 PENNSYLVANIA AVENUE, N.W., SUITE 800, WASHINGTON, DC, 20037, US

(73) Assignee: COMPUMEDICS MEDICAL INNOVATION PTY LTD, Abbotsford (AU)

(21) Appl. No.: 12/680,916

(22) PCT Filed: Sep. 30, 2008
(86) PCT No.: PCT/AU2008/001445

§ 371 Date: Mar. 31, 2010

(30) Foreign Application Priority Data
Oct. 2, 2007 (AU) 2007905367

(51) Int. Cl. A61B 5/0205

(52) U.S. Cl. 600/484

(57) ABSTRACT

The present invention provides a method and apparatus for determining the occurrence of apnoeas or hypopneas from ECG signal data alone. The method is carried out by apparatus configured to acquire ECG signals from a sleeping subject, transform the signals to data, and extract ECG features relevant to estimate breathing effort for the determination of respiratory events characteristic of apnoeas and hypopneas. The extracted ECG features are correlates of breathing efforts and are used as surrogate measures of breathing or respiratory events. The method may include calculating an AHI or apnoea/hypopnea index. The method may classify apnoeas into obstructive or central apnoeas.

### FIELD OF THE INVENTION

This invention relates to methods and apparatus for acquiring and analysing electrocardiograph data from a subject, particularly from a subject having sleep-disordered breathing, more particularly, sleep apnoea, even more particularly, obstructive sleep apnoea or central sleep apnoea.

### BACKGROUND

Sleep apnoea (SA), including obstructive sleep apnoea (OSA) and/or central sleep apnoea (CSA), in which breathing is arrested, affects a significant proportion of the population. The effects of OSA are felt when the airways collapse and air cannot pass. The effects of CSA are felt when a subject does not inspire and expire air due to causes associated with the functioning of the central nervous system. Both OSA and CSA can result in inefficient sleep and further medical problems.

Both OSA and CSA are currently diagnosed in sleep laboratories by overnight polysomnographic (PSG) studies where a subject sleeps while having numerous electrodes attached to the body for measuring various physiological parameters. Such PSG studies are costly to undertake because they require a subject to sleep overnight in a clinic.

Some approaches have been made to develop apparatus and methods for measuring the occurrence and type of SA. Standard PSG rules for diagnosing SA require detection of the actual start times, durations and categories of individual respiratory events. This information is then used for evaluating the presence and severity of sleep apnoea by generating an apnoea-hypopnea index (AHI) [1]. Currently the most successful reported methods can detect if a subject has sleep apnoea (without reliable estimate of the AHI value) or if there is a sleep apnoea event during any given minute [2-5]. For example, U.S. Pat. No. 5,769,084 teaches a method of using chaotic processing of various sleep parameters, including measuring the electrocardiogram (ECG), for determining the presence of SA. However, the method does not extend to discriminating OSA from CSA. A method for measuring SA from sensors located on different planes on a subject is taught in U.S. Pat. No. 6,415,174. This document does not teach a method that can discriminate between CSA and OSA. In WO 2005/067790, incorporated herein by reference, Burton indicated that it was possible to use an ECG trace to identify instances of SA and classify the SA as either OSA or CSA. However, the CSA signal is small relative to the background noise, i.e. there is a small signal-to-noise ratio.

There is a large prevalence of OSA and CSA among cardiac patients making the use of ECG data to determine the AHI an even more attractive option than the use of PSG data. What is needed is a more convenient and reliable method to measure sleep apnoea and its form using simpler apparatus than PSG studies. The methods should be able to detect and classify individual respiratory events. Preferably, such methods would be carried out in the home [6-8].

### BRIEF DESCRIPTION OF THE FIGURES

FIG. 1 shows an overview flow chart of the steps for calculating the hypopnea/apnoea index from ECG data complexes.

FIG. 2 shows a flow chart of the detailed steps for calculating the hypopnea/apnoea index from ECG data complexes.

FIG. 3 shows a graph of the output of a typical ECG lead I complex with each section of the graph labelled.

FIG. 4 shows a plot of ECG signal against time of the QRS area, R-amplitude, and respiration flow rate of a subject displaying regular, steady breathing during sleep.

FIG. 5 shows a plot of ECG signal against time of the QRS area, R-amplitude, and respiration flow rate of a subject displaying a respiratory event.

FIG. 6 shows a graph of mV of the R-wave against QRS complex number in an ECG signal in a respiratory event during sleep.

FIG. 7 shows a graph of mV of the R-R interval and flow rate of respiration against time against QRS complex number in an ECG signal over a series of respiratory events during sleep.

FIG. 8 shows a graph of the signals against time for QRS area, R-amplitude in an ECG signal, and flow rate during periods of apnoea and normal breathing during sleep.

FIG. 9 shows a graph of amplitude of the R-wave against QRS complex number in an ECG signal during a respiratory event.

FIG. 10 shows a graph of area of QRS region against QRS complex number in an ECG signal during a respiratory event.

FIG. 11 shows a graph of R-R interval against QRS complex number in an ECG signal over a respiratory event.

FIG. 12 shows a graph of QRS area against QRS complex number through a respiratory event in an ECG signal.

FIG. 13a shows the output signal showing the R-wave though a respiratory event in a first ECG lead.

FIG. 13b shows the output signal showing the R-wave from a second ECG lead through the respiratory event shown in FIG. 12a.

FIG. 14 shows a graph of R-R interval against QRS number for an ECG signal through a respiratory event.

FIG. 15 shows a plot of EDR signal against QRS complex number in an ECG signal through a respiratory event.

FIG. 16 shows a graph of the amplitude of an R-wave against QRS complex number in an ECG signal through a respiratory event.

FIG. 17a shows graphs of QRS area and R-wave amplitude in an ECG signal over time for an OSA event.

FIG. 17b shows graphs of QRS area and R-wave amplitude in an ECG signal over time for a CSA event.

### SUMMARY OF THE INVENTION

The present invention exploits the surprising observation that ECG-derived parameters are correlated with respiratory events and the ECG-derived correlates may be used to identify respiratory events, in particular, in sleeping subjects. The identified respiratory events may be used to determine respiratory effort. The determination of respiratory effort may be used for calculating the apnoea/hypopnea index. The ECG-derived data may also be used to discriminate between OSA and CSA. It is an object of the invention to provide a method for identifying respiratory events using ECG signal data. It is a further object of the invention to provide a method for determining respiratory effort using ECG signal data. It is a further object of the invention to provide a method for calculating an apnoea/hypopnea index from ECG signal data.

In one aspect, the invention provides a method for determining respiratory events from an ECG signal, comprising the steps of: acquiring ECG signal data; extracting waveform morphology data from said ECG signal data; and estimating the respiratory effort from said waveform morphology data. The method may include the step of cross validating the peaks of the respiratory effort. The method may include the steps of characterising breathing patterns from said respiratory effort and detecting the respiratory events for observation. The method may include the step of classifying a respiratory event as one of apnoea or hypopnea. The method may include the step of classifying a respiratory event as one of obstructive apnoea or central apnoea. The method may include the step of calculating an apnoea/hypopnea index. The waveform data may be transformed into any one or combination of ECG-derived physiological predictors including ECG-derived respiration signal, heart rate, or area or amplitude or periodicity of the R-signal. Preferably, the method of is practised on a subject in a sleep state.

In a further aspect, the invention provides a method for analysing sleep-disordered breathing comprising the steps of: acquiring biosignals from ECG sensors; storing the biosignals as data in a computer file; extracting heart rate and waveform morphology data from said biosignal data; deriving physiological predictors from extracted heart rate and ECG waveform morphology data; and determining the start and end of respiratory events from said derived physiological predictors. The method may include calculating the apnoea/hypopnea index from the average number of respiratory events per hour that are longer than ten seconds. The method may include displaying the apnoea/hypopnea index.

In a still further aspect, the invention provides apparatus for determining respiratory effort from an ECG signal comprising: at least two ECG leads for acquiring at least two orthogonal signals from a subject; means for transforming said signals to digital data; electronic data storage means; and a microprocessor programmed to extract waveforms from digital ECG data and determine respiratory events and respiratory effort. The apparatus may further comprise of a microprocessor programmed to calculate an apnoea/hypopnea index.

### DESCRIPTION OF THE INVENTION AND MOST PREFERRED EMBODIMENT

The present invention provides a previously unknown method for determining an apnoea/hypopnea index from ECG signal data acquired from a subject. The method of the invention advantageously exploits the variation in ECG signals acquired during sleep to calculate an apnoea/hypopnea index for the period of sleep.

Different ECG leads measure a difference in electro-potentials across different regions of the body. Breathing affects the impedance between electrodes and therefore the potentials and the differences among them. The movement of the chest and abdomen changes during respiratory events and therefore changes how the impedance between electrodes changes with breathing effort. This results in a phase change between ECG derived signals that can be observed.

The method uses the phase changes as markers for respiratory events. The method advantageously exploits the short period after a respiratory event and during an arousal where there is short period of increased heart rate, systolic blood pressure, and diastolic blood pressure accompanied by increased sympathetic activity. Apnoea events are usually terminated with an arousal enabling the markers of an arousal to be used as markers for apnoea termination. The burst of increased heart rate is easily detectable from the ECG. Sudden drops in the R-R interval as described below are used as markers for the end of respiratory events.

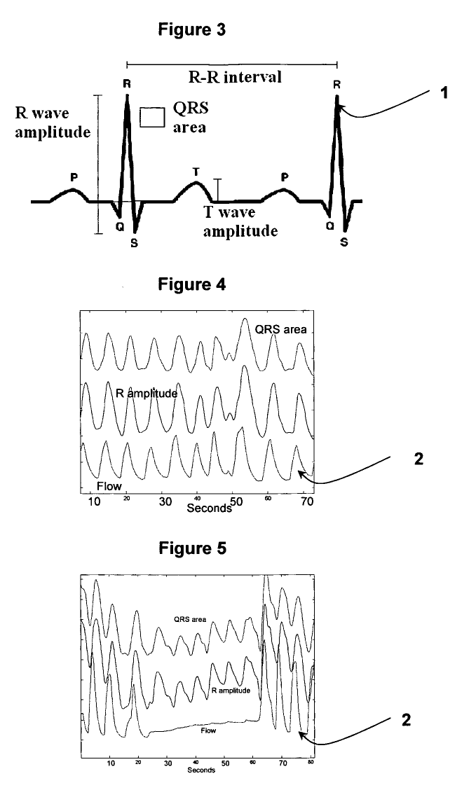

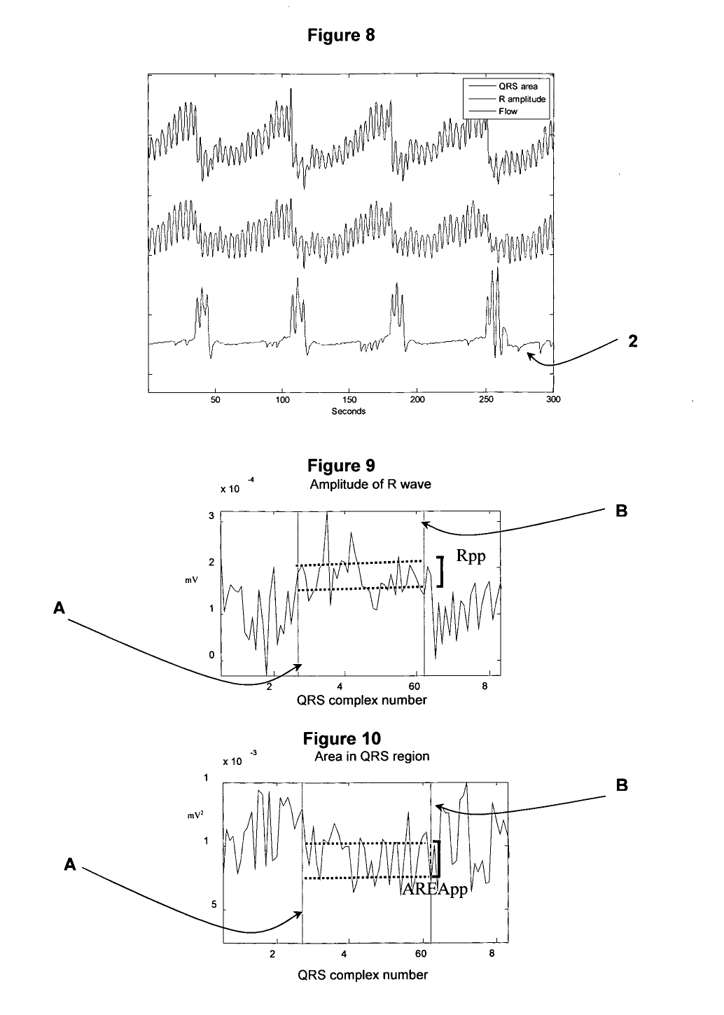

Sleep apnoea is often cyclic in nature and this can result in many of the extracted ECG signals also presenting a cyclic pattern as well which is shown particularly in the lowest trace of flow rate 2 of expired air in FIG. 8. It can be observed that the QRS area and R wave amplitude also have a periodic pattern of the same frequency. (The parameters of an ECG signal are shown as the trace 1 in FIG. 3.) As the breathing signal develops a longer term periodic pattern the accompanying extracted ECG signal also develops a periodic pattern with the same frequency, which may be used as surrogate estimated of the breathing signal in the method of the invention. Fourier analysis techniques and smoothing techniques using digital filtering may be used in the method to recognise these patterns. Envelopes that are found to contain these cyclic patterns are used to highlight regions of the data for closer examination as explained herein.

The method identifies the start and end points of respiratory events which have characteristics measurable in ECG signal data. Most conveniently, the ECG signal data are acquired by sensors adjacent a subject while the subject is asleep. The data is collected and stored in a computer file for manipulation with a microprocessor and computer programs to identify respiratory events, determine the respiratory effort, or calculate the apnoea/hypopnea index.

Table 1 provides definitions for some the parameters measurable in the ECG signal data and their acronyms used in this description. The parameters will be well known by persons skilled in the art of acquiring and analysing ECG signals. The method is not limited to the parameters in Table 1, but may include any ECG-derived signal which has a correlation with breathing effort.

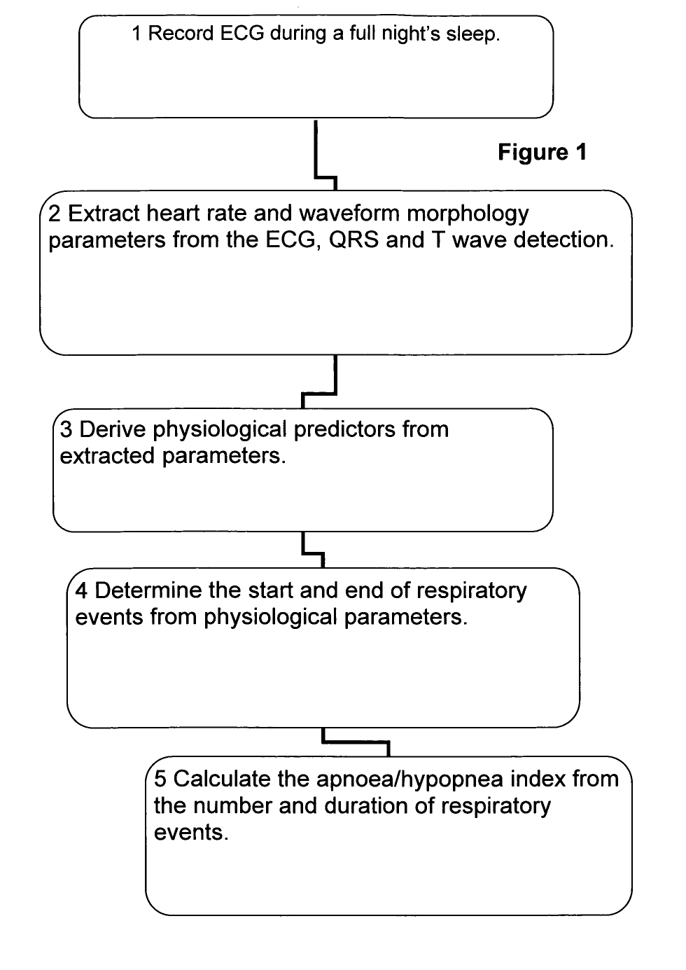

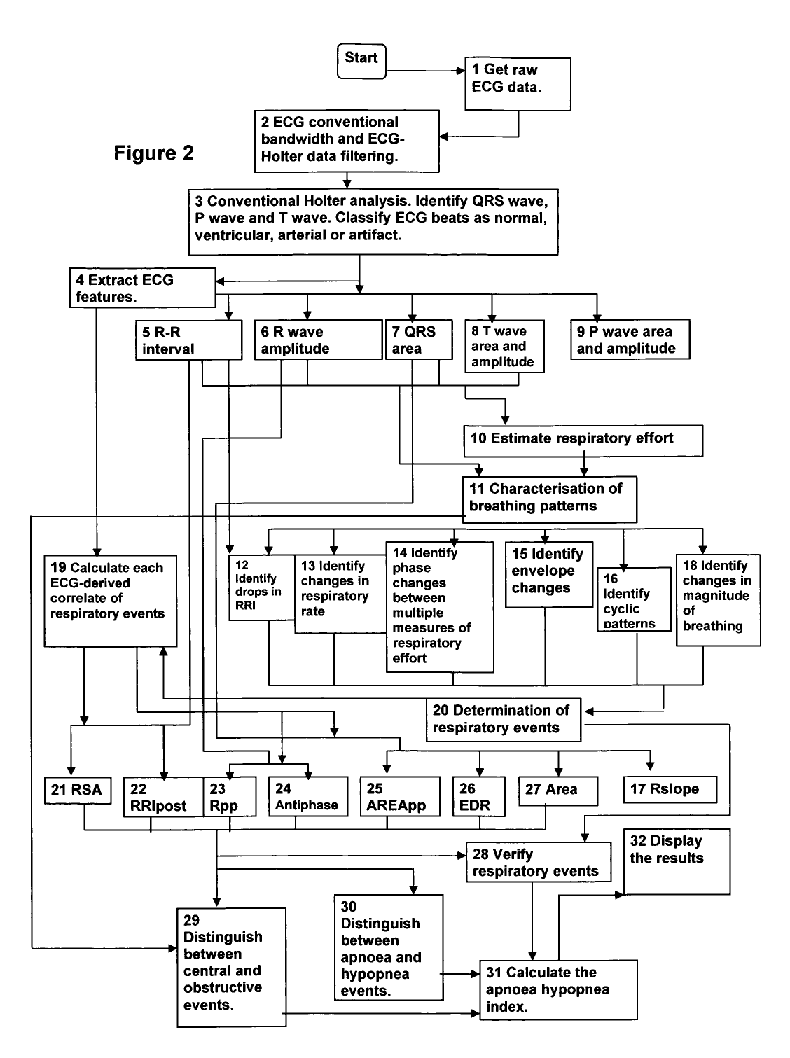

An overview of the steps of the method of the invention is presented in the flow-chart of FIG. 1. A more detailed flow-chart further detailing the method is shown in FIG. 2. A typical ECG trace 1 is shown in FIG. 3 as a plot of acquired signal magnitude against time. All calculations of the method are carried out using a microprocessor. Referring to FIG. 1, in a first step, the ECG signal is recorded from a subject using standard ECG monitoring equipment such as an ECG holter. Any suitable equipment that can record a digital ECG signal may be used. The acquired data is conveniently stored in a digital file on computer media for analysis. The data may be filtered according to a relevant bandwidth for ECG data using known techniques.

The ECG signal 1 over time has a characteristic wave-form morphology incorporating segments, shown in FIG. 3, which are identified in the second step of the method. In particular, the peak commencing at point Q, reaching a maximum at R, and a minimum at S, is well known as the “QRS” complex. Further parameters used in the method and shown in the Figures are the amplitude and area under the graph of the T wave of each ECG complex, the amplitude of the signal at R, the time period between successive R waves, and the area under the graph of the QRS segment. The heart rate is measured as the time between R wave peaks shown in FIG. 3 is known as the R-R interval (RRI).

The ECG-derived signals are used to generate a number ECG-derived correlates of respiratory events in a third step. These correlates are used to confirm changes that occur in the ECG during periods of respiratory or breathing events, including apnoea or hypopnea events. Each of the correlates that may be used in the method is described herein. The scope of the invention includes the use of any ECG-derived parameter that correlates with breathing events. The identified ECG parameters are then classified as being representative of normal or abnormal heart function. This analysis uses linear approximation to identify the various regions of the ECG complex. However, other relevant analyses may be used. The classification is carried out by examining changes to the ECG parameters identified in the second step.

The classification of a parameter as being normal or abnormal uses known correlations. For the classification of the heart rate, the method incorporates the following correlation. Respiratory Sinus Arrhythmia (RSA) is a naturally occurring rhythm observed in the heart rate (HR) or R-R interval (RRI) of the ECG, in mammals. This rhythm is the direct result of the interactions of the respiratory and the cardiovascular system. RSA is characterized by a periodic signal that displays maxima and minima in the RRI which is similar to the respiratory rate. Typically, the HR will increase with inspiration and decrease with expiration. Changes to the RSA signal may be used as a physiological predictor in the method.

It is well known that after a respiratory event there is a decrease in the RRI. This is associated with an increase in sympathetic activity during arousal. During an arousal there is a short period of increased heart rate, systolic blood pressure, and diastolic blood pressure accompanied by increased sympathetic activity. The heart rate variability is also reduced by the increase in sympathetic activity. This reduction in RRI may be used as a physiological predictor in the method.

The morphology of the ECG waveform is used to classify whether or not respiration is normal according to changes with respiration due to the motion of the heart with respect to the electrodes correlating with the changing intra-thoracic impedance. The rotation of the heart with respect to the electrodes, caused by breathing is evident in oscillations of both the amplitude of the R wave and the area of the R wave (this included the area of the whole QRS segment shown in FIG. 3). As breathing is reduced or stops completely during an event a reduction in the peak to peak oscillations of the signal is expected. Changes to the amplitude and area may be used as physiological predictors in the method.

A sudden shift in orientation of the heart will cause a sudden change in the amplitude of the R wave. If the ECG leads are orthogonal the shift should be in the opposite directions. The opposite change in amplitude of the R wave in ECG leads may be used as a physical predictor in the method.

A gradual increase in the amplitude of the R or T wave may be used as a physical predictor. An example of an increasing R wave in an ECG signal is shown in FIG. 16.

In a fourth step, by examining the changes in the physiological predictors for each ECG complex the method determines if each of the ECG complexes occurs at the start of a respiratory event. The start and end of each respiratory event is then determined in the fourth step of the method. FIG. 2 shows the steps of the method for calculating the apnoea/hypopnea index from successive ECG complexes stored in a data file.

In a fifth step, the number of detected respiratory events and their duration is used calculate the apnoea/hypopnea index. The apnoea/hypopnea index is calculated as the average number of respiratory events that are longer than ten seconds, per hour.

The method advantageously uses ECG signal data alone to characterise breathing patterns. By examining the ECG derived respiratory effort and the extracted ECG signals, regions of data that contain changes in breathing patterns associated with respiratory events are identified. There are a number of changes described herein that may be investigated to help identify possible respiratory events.

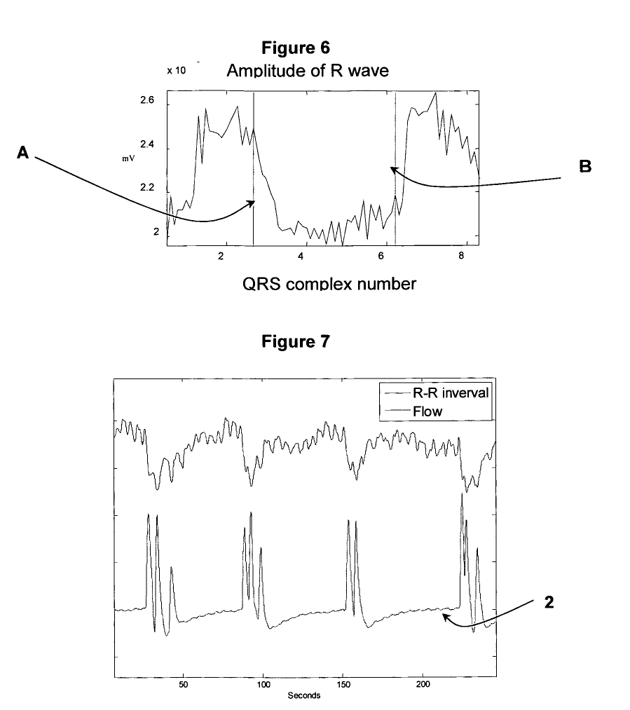

FIG. 5 shows a graph of ECG signal parameters, QRS area and R-amplitude as well as the measured airflow 2 over an obstructive sleep apnoea event. There is expected to be a reduction in breathing magnitude during the event compared to before and after the event, which is clearly shown in FIG. 5. Possible respiratory events are identified by examining the respiratory effort and locating regions that have a reduction in respiratory effort. The method uses identification of changes in the breathing rate to assist in marking possible respiratory events, i.e., the changes to the breathing rate that occur on event onset, during the event and on arousal. The method may use the sharp change in the in the QRS area and R wave amplitude occurs and the beginning and end of respiratory events. A sudden shift in orientation of the heart will cause a sudden change in the amplitude of the R wave. Sudden shift in the ECG-derived signals are used to mark the possible beginning and end of respiratory events. This is shown in FIG. 6, where the sudden shift that occurs during a respiratory event is evident in this segment if the R wave amplitude signal. The lines at A and B mark the beginning and end of the respiratory event respectively. FIG. 7 shows the dips in the RRI signal can be observed after each obstructive apnoea event, evident in the flow.

The method may include determination of breathing patterns from the ECG-derived measures of respiratory effort. This may include patterns of reduction in the magnitude of respiratory effort as shown in FIG. 5 or patterns of abrupt variation in the respiratory rate as shown in FIG. 7 or patterns of abrupt variation in the underlying level of respiratory effort markers shown in FIG. 6. The method may use patterns of change in phase relationships between multiple measures of respiratory efforts, patterns of consistent variation in the magnitude of respiratory effort estimate(s) over the course of a sequence of breaths, or cyclical patterns in the envelope of respiratory effort estimate(s) as shown in FIG. 8.

More specifically, with reference to FIG. 2 raw ECG data is obtained from the subject via a two-lead (or greater) ECG recording. This must use at least two ECG leads that are orthogonal. This data is saved to a digital storage device. The calculation of the AHI commences with a microprocessor programmed to access and analyse the stored ECG signal data (Box 1 FIG. 2) and filtering, if necessary, for the relevant bandwidth (Box 2 FIG. 2). It will be understood that the large amount of signal data acquired and transformations required is carried out with a microprocessor with processing speed and power known in the art to be required, but also programmed to acquire, store, and manipulate signals and data according to the method described herein.

The analysis of the stored data commences with the examination using conventional Holter analysis programs and the QRS wave, P wave and T wave segments of the ECG are identified (Box 3 FIG. 2). Each segment of the heart beat is then classified as normal, ventricular, arterial or artefact. An example of the collected data is graphed in FIG. 4, which shows a representation of extracted ECG features from a sleeping subject breathing normally. The signal displayed against time in FIG. 4 is that acquired from lead I of an ECG lead configuration known in the art. By examining properties of the identified waves ECG derived signals are generated by the software. The generated ECG derived signals are; R wave amplitude, QRS area, R-R interval (RRI), T wave amplitude, and T wave area. The first two signals, as well as the measured respiratory airflow are shown in FIG. 4. The software program extracts morphological features of the ECG signal(s) (Box 4, FIG. 2) including: start times, end times and peak times for the P-waves, QRS complexes and T-waves by means of morphological analysis of the ECG signal(s). This includes classifying QRS complexes as normal, ventricular, supraventricular or artifact by means of applying arrhythmia classification rules. Generating quantitative parameters of the ECG signal(s) including R-R intervals, R-wave amplitude, QRS complex area, T-wave amplitude and T-wave area (Boxes 5-9, FIG. 2).

Many of the ECG derived signals demonstrate oscillatory patterns similar to the oscillatory patterns that occur with breathing (FIG. 4). The breathing effort is estimated from the QRS area and R wave amplitude which oscillate with breathing (Boxes 10-11, FIG. 2). The breathing signal, or ECG-Derived Respiration signal (EDR) may be calculated from the ratio of the areas of the QRS segment from orthogonal ECG leads (Box 26 FIG. 2).

Local peaks that are associated with changes in breathing direction (changing from exhalation to inhalation or vice versa) are identified in each of the ECG derived signals (Box 10 FIG. 2). These peaks are identified by examining oscillatory patterns, similarity of periods and signal magnitudes for respiratory peak candidates within there ECG derived signals.

Once these peaks have been identified in each of the ECG derived signals they are compared between signals. This enables a cross validation of the respiratory peaks in each signal (Box 10 FIG. 2). By examining and comparing each of the peaks in each signal the single set of respiratory peaks are identified. This achieved by comparing the period between each of the peaks and the changes in amplitude of the peaks between signals (Box 10 FIG. 2). By examining; the changes in the ECG derived signals and the estimated respiratory effort using the methods described above, markers for the beginning and end of possible ECG events are identified. These markers of possible beginning and end of events are used to generate a set of respiratory event candidates. The respiratory events are compared with the pre-defined or adjustable rules for the detected patterns of estimated breathing and R-R intervals including application of look-up tables and artificial neural networks.

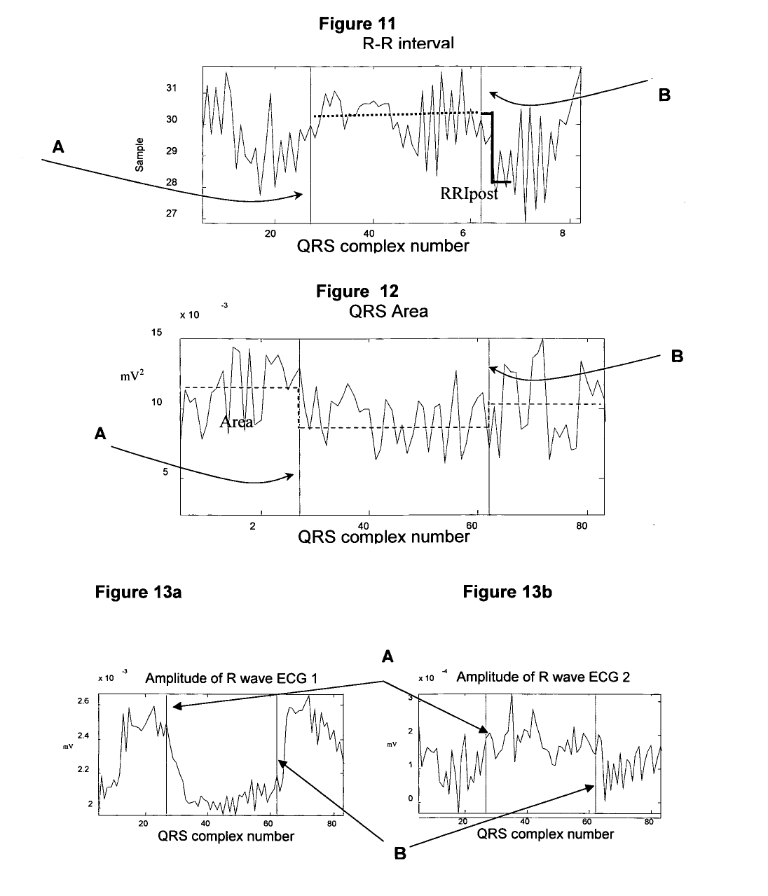

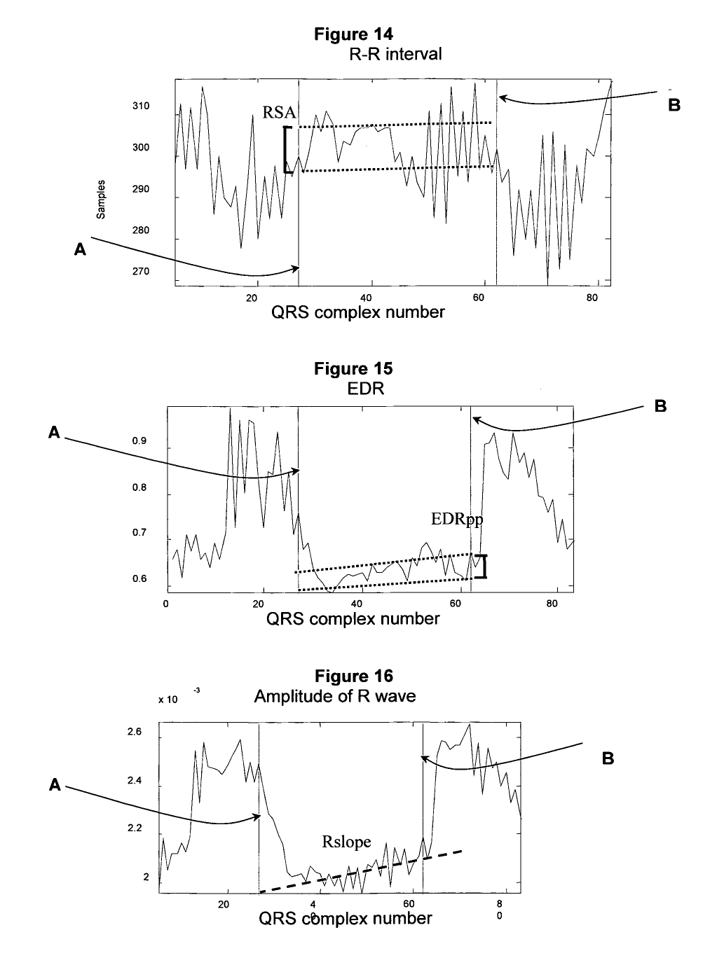

The method calculates the following parameters. The Reduction in the average peak to peak magnitude of oscillations of the R wave amplitude over the course of a potential respiratory event relative to the respective values before and after the event is shown in FIG. 9. The reduction in the average peak to peak magnitude of oscillations of the area under the QRS complex over the course of a potential respiratory event relative to the respective values before and after the event is also shown in FIG. 10. The reduction in the average R-R interval after a potential respiratory event relative to the average R-R interval over the course of the event is shown in FIG. 11. The variation in the average value of the area under QRS complex over the course of potential respiratory event relative to respective values before and after the event is shown in FIG. 12. The difference between variations in the average values of R wave amplitude over the course of potential respiratory event relative to the respective values before and after the event for pairs of multiple ECG signals as shown in FIG. 13. The reduction in the average peak to peak magnitude of oscillations of R-R interval over the course of potential respiratory event relative to the respective values before and after the event as shown in FIG. 14. The reduction in the average peak to peak magnitude of the ratio of areas under QRS complexes detected from the two orthogonal ECG leads over the course of potential respiratory event relative to the respective values before and after the event as shown in FIG. 15. The estimate of linear trend for the R wave amplitude by means of fitting the least squares error linear approximation over the course of potential respiratory event is shown in FIG. 16.

For each of the respiratory event candidates each of the ECG derived correlates of respiratory events are generated (Boxes 17, 19, 21-27 FIG. 2). These correlates are used to either confirm or reject each of the respiratory event candidates. The respiratory event candidates that demonstrate that ECG derived correlates of respiratory events that generate the expected are accepted.

Once the respiratory peaks have been verified they can be used to generate an estimation of respiratory effort (Box 28 FIG. 2). The respiratory effort is estimated by interpolating the sequences of values of R-wave amplitudes and QRS-complex areas into respective channels for from single or multiple ECG signals. These interpolated channels serve as approximate markers of respiratory effort. Sequences of respiratory peaks from individual approximate markers of respiratory effort are determined by means of detecting peaks on those markers with similar distances, signal variations, and slopes. Fine tuning sequences of respiratory peaks by means of matching the peak sequences determined from the individual markers to eliminate false positive respiratory peaks and recover missing respiratory peaks.

The rotation of the heart with respect to the electrodes, caused by breathing is evident in oscillations of both the amplitude of the R wave and the area of the R wave (this included the area of the whole QRS segment). As breathing is reduced or stops completely during an event a reduction in the peak to peak oscillations of the signal would be expected. As shown in FIGS. 9 and 10, the Rpp and the AREApp correlates are calculated by finding the value of the mean peak to peak oscillations of the R wave amplitude/QRS area during the event minus the value of the mean peak to peak oscillations of the R wave amplitude/QRS area before and after the event. FIG. 9 shows the reduction in oscillations can be seen in the amplitude of the R wave during the respiratory event. The lines, A B, respectively, mark the beginning and end of the respiratory event. FIG. 10 shows the reduction in oscillations can be seen in the area of the QRS segment during the respiratory event. The lines, A B, respectively, mark the beginning and end of the respiratory event.

It is well known that after a respiratory event there is a decrease in the RR-interval. This is associated with an increase in sympathetic activity during arousal. During an arousal there is a short period of increased heart rate, systolic blood pressure, and diastolic blood pressure accompanied by increased sympathetic activity. The heart rate variability is also reduced by the increase in sympathetic activity. The difference between the mean value of the RRI during the event and the mean value of the RRI after the event was used to calculate the RRI_post correlate which may be used in the method. FIG. 11 shows the reduction in the R-R interval directly after respiratory event can be observed. The lines A, B, respectively, mark the beginning and end of the respiratory event.

A shift in orientation of the heart causes a change in the QRS area and R wave amplitude. The difference between the mean QRS area during the event and the mean QRS before and after the event is used to calculate the AREA correlate. FIG. 12 shows the change in mean QRS area during the respiratory event. The lines, A, B, respectively mark the beginning and end of the respiratory event.

The difference between the mean R wave amplitude during the event and the mean R wave amplitude directly preceding and succeeding the event was calculated for two channels and then multiplied together to calculate the Antiphase correlate. If the ECG leads are orthogonal the shift should be in the opposite directions.

FIG. 13 shows a sudden change in R wave amplitude being evident and the change in each lead is in opposite directions. The lines, A, B, respectively mark the beginning and end of the respiratory event.

Respiratory Sinus Arrhythmia (RSA) is a naturally occurring rhythm observed in the heart rate (HR) or R-R interval (RRI) of the ECG. This rhythm is the direct result of the interactions of the respiratory and the cardiovascular systems. RSA is characterized by a periodic signal that displays maxima and minima in the RRI which is similar to the respiratory rate. Typically the HR will increase with inspiration and decrease with expiration. As demonstrated in, the difference of the value of the mean peak to peak oscillations of the RRI during the event and the value of the mean peak to peak oscillations of the RRI before and after the event was used to calculate the RSA correlate.

FIG. 14 shows the reduction in oscillations of the R-R interval during the respiratory event can be observed. The lines A, B, respectively mark the beginning and end of the respiratory event.

The ECG Derived Respiration signal (EDR) is calculated as the ratio of the areas of the QRS segment from orthogonal ECG leads to determine a respiratory signal. The difference of the mean peak to peak oscillations of the EDR signal during the event and the value of the mean peak to peak oscillations of the EDR signal before and after the event is used to calculate the EDRpp correlate. FIG. 15 shows the reduction in oscillations of the EDR signal during the respiratory event can be observed. The lines A, B, respectively, mark the beginning and end of the respiratory event.

FIG. 16 shows an example ECG signal data where during the event there was a gradual increase in the amplitude of the R waves. The slope of the linear best fit, shown by the dashed line, for the R wave amplitude during the event may be used to calculate the Rslope correlate.

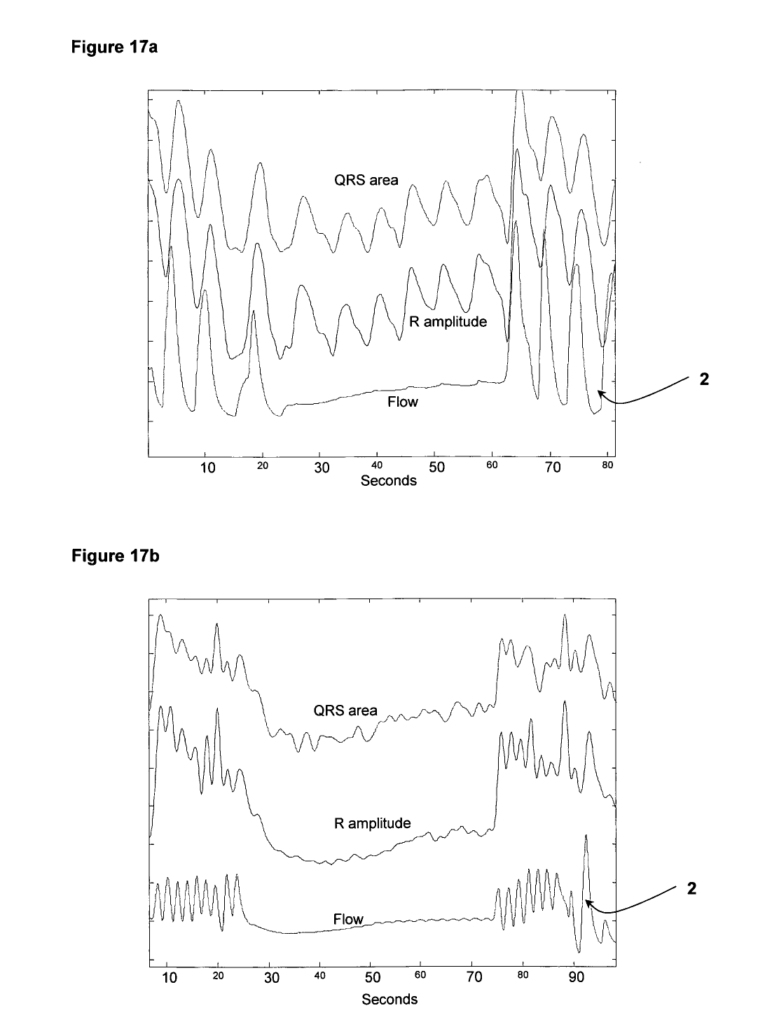

The respiratory effort from the QRS area and R wave amplitude enable event classification. As these signals can be used to estimate respiratory effort they can be used to assist in distinguishing between central events and obstructive events (Box 29 FIG. 2). Respiratory effort should be evident and possibly increase during an obstructive event, while during a central event the respiratory effort should remain very low or nonexistent. Examples of this are presented in FIGS. 17a and b, which show differences between ECG parameters in OSA and CSA events. FIG. 17a how the QRS area and R wave change during an obstructive event. It is evident that respiratory effort remains during the event. FIG. 17b shows the QRS area and R wave change during a CSA event. It is evident that respiratory effort is dramatically reduced or non-existent. Also, ECG derived correlates of respiratory events yield different results depending on the type of event examined. For example the AREApp correlate would expect to have a large magnitude for a central event than an obstructive event as there should be a greater reduction in the oscillations of the QRS area that are correlated with breathing.

As there is a greater reduction in breathing during apnoea events compared to hypopnea events, larger changes in ECG derived correlates of respiratory events are expected in apnoea events than with hypopnea events. By quantifying these changes in ECG derived correlates of respiratory events the method may discriminate between apnoea and hypopnea events. The method may discriminate between apnoea and hypopnea events by calculating the ratios of average oscillation magnitudes within the respiratory events to the respective values before and after respiratory events for the ECG derived measures of respiratory effort with the events that have these ratios below the predetermined thresholds being classified as apnoeas and the other events classified as hypopneas.

The AHI is calculated dividing the total number of respiratory events by the number of hours of ECG signal recording period (Box 31 FIG. 2). Calculating the apnoea/hypopnea index for the sleep evaluation time interval provides a clinical estimate of the presence, severity and type of sleep apnoea by means Separate indices may being calculated for obstructive, central and combination of obstructive and mixed respiratory events. Preferably, the index is displayed on a suitable monitor for viewing by a user of the method.

TABLE 1
SUMMARY OF THE DESCRIPTION OF ECG DERIVED
CORRELATES OF RESPIRATORY EVENTS

| Description of ECG derived correlates of | Physiological |
|---|---|
| respiratory events | Predictor |
| Change in peak to peak oscillations of the EDR signal | EDR |
| during the event. | |
| Measure of the sudden opposite change of R wave | Antiphase |
| amplitude between 2 orthoganal ECG leads during | |
| the event. | |
| Change in RSA signal (peak to peak oscillations of the | RSA |
| RRI signal). | |
| Change in the mean RRI signal after the event. | RRIpost |
| Change in peak to peak oscillations of the R amplitude | Rpp |
| during the event. | |
| Change in peak to peak oscillations of the QRS area | AREApp |
| during the event. | |
| The change in the QRS area during the event. | Area |
| Measure of the increase in amplitude of the R wave | Rslope |
| during the event. | |

### REFERENCES

[1] “Sleep-related breathing disorders in adults: recommendations for syndrome definition and measurement techniques in clinical research. The Report of an American Academy of Sleep Medicine Task Force,” Sleep, vol. 22, pp. 667-89, Aug. 1, 1999.
[2] M. Schrader, C. Zywietz, V. von Einem, B. Widiger, and G. Joseph, “Detection of sleep apnea in single channel ECGs from the PhysioNet data base,” 2000, pp. 263-266.
[3] C. Maier, H. Dickhaus, M. Bauch, and T. Penzel, “Comparison of heart rhythm and morphological ECG features in recognition of sleep apnea from the ECG,” in Computers in Cardiology, 2003, 2003, pp. 311-314.
[4] S. Vijendra, K. Behbehani, E. A. Lucas, J. R. Burk, D. N. Burli, and D. H. Dao, “The use of R-wave morphology in the detection of sleep-disordered breathing using the electrocardiogram—a comparison between leads,” 2004, pp. 3881-3884 Vol. 6.
[5] F. Roche, V. Pichot, E. Sforza, I. Court-Fortune, D. Duverney, F. Costes, M. Garet, and J. C. Barthelemy, “Predicting sleep apnoea syndrome from heart period: a time-frequency wavelet analysis,” Eur Respir J, vol. 22, pp. 937-42, December 2003.
[6] J. D. Lattimore, D. S. Celermajer, and I. Wilcox, “Obstructive sleep apnea and cardiovascular disease,” J Am Coll Cardiol, vol. 41, pp. 1429-37, May 7, 2003.
[7] J. Wolf, J. Lewicka, and K. Narkiewicz, “Obstructive sleep apnea: An update on mechanisms and cardiovascular consequences,” Nutr Metab Cardiovasc Dis, Feb. 19, 2007.
[8] M. T. Naughton, “Heart failure and obstructive apnoea,” Sleep Medicine Reviews, vol. 2, pp. 93-103, 1998.
[9] P. Grossman and E. W. Taylor, “Toward understanding respiratory sinus arrhythmia: relations to cardiac vagal tone, evolution and biobehavioral functions,” Biol Psychol, vol. 74, pp. 263-85, February 2007.
[10] J. W. Denver, S. F. Reed, and S. W. Porges, “Methodological issues in the quantification of respiratory sinus arrhythmia,” Biol Psychol, vol. 74, pp. 286-94, February 2007.
[11] F. Yasuma and J. Hayano, “Respiratory sinus arrhythmia: why does the heartbeat synchronize with respiratory rhythm?,” Chest, vol. 125, pp. 683-90, February 2004.
[12]H. J. Burgess, J. Kleiman, and J. Trinder, “Cardiac activity during sleep onset,” Psychophysiology, vol. 36, pp. 298-306, May 1999.
[13] M. J. Carrington, R. Barbieri, I. M. Colrain, K. E. Crowley, Y. Kim, and J. Trinder, “Changes in cardiovascular function during the sleep onset period in young adults,” J Appl Physiol, vol. 98, pp. 468-76, February 2005.
[14] R. J. Davies, P. J. Belt, S. J. Roberts, N. J. Ali, and J. R. Stradling, “Arterial blood pressure responses to graded transient arousal from sleep in normal humans,” J Appl Physiol, vol. 74, pp. 1123-30, March 1993.
[15] K. Narkiewicz, N. Montano, C. Cogliati, P. J. van de Borne, M. E. Dyken, and V. K. Somers, “Altered cardiovascular variability in obstructive sleep apnea,” Circulation, vol. 98, pp. 1071-7, Sep. 15, 1998.
[16] G. B. Moody, R. G. Mark, A. Zoccola, and S. Mantero, “Derivation of Respiratory Signals from Multi-lead ECGs,” Computers in Cardiology, vol. 12, pp. 113-116, 1985.

1. A method for determining respiratory events from an ECG signal, comprising
the steps of acquiring ECG signal data;
extracting waveform morphology data from said ECG signal data; and estimating the respiratory effort from said waveform morphology data.

2. The method of claim 1 further comprising the step of cross validating the peaks of the respiratory effort.

3. The method of claim 1, further comprising the steps of characterising breathing patterns from said respiratory effort; and detecting the respiratory events for observation.

4. The method of claim 1, further comprising the step of classifying a respiratory event as one of apnoea or hypopnea.

5. The method of claim 1, further comprising the step of classifying a respiratory event as one of obstructive apnoea or central apnoea.

6. The method of claim 4, further comprising the step of calculating an apnoea/hypopnea index.

7. The method claim 1 wherein said waveform data is transformed into any one or combination of ECG-derived physiological predictors including ECG-derived respiration signal, heart rate, or area or amplitude or periodicity of the R-signal.

8. The method of claim 1 practised on a subject in a sleep state.

9. A method for analysing sleep-disordered breathing comprising the steps of:
acquiring biosignals from ECG sensors adjacent a subject;
storing the biosignals as data in a computer file;
extracting heart rate and waveform morphology data from said biosignal data;
deriving physiological predictors from extracted heart rate and ECG waveform morphology data; and
determining the start and end of respiratory events from said derived physiological predictors.

10. The method of claim 9 further comprising the step of classifying a respiratory event as an apnoea or a hypopnea.

11. The method of claim 9 further comprising the steps of calculating the apnoea/hypopnea index; and
displaying the apnoea/hypopnea index.

12. Apparatus for determining respiratory effort from an ECG signal comprising:
at least two ECG leads for acquiring at least two orthogonal signals from a subject;
means for transforming said signals to digital data;
electronic data storage means; and
a microprocessor programmed to extract waveforms from digital ECG data and determine respiratory events and respiratory effort.

13. Apparatus for determining respiratory effort according to claim 12 further comprising a microprocessor programmed to calculate an apnoea/hypopnea index.
